(19) United States
(12) Patent Application Publication
(10) Pub. No.: US 2026/0010199 A1
(43) Pub. Date: Jan. 8, 2026

(54) TOKEN SMART RING FOR WIRELESS PAYMENT AND HEALTH MONITORING

(71) Applicant: Ka Wai Wayne LEUNG, Hong Kong (CN)

(72) Inventor: Ka Wai Wayne LEUNG, Hong Kong (CN)

(21) Appl. No.: 19/105,611

(22) PCT Filed: Sep. 7, 2022
(86) PCT No.: PCT/IB2022/058423

§ 371 Date: Feb. 21, 2025

(51) Int. Cl. G06F 1/16; G06Q 20/32; G16H 40/67

(52) U.S. Cl. CPC G06F 1/163; G06Q 20/321; G06Q 20/3278; G16H 40/67

(57) ABSTRACT

A token smart ring, preferably adapted for wireless payment and/or health monitoring, including a main portion adapted for engaging with an external associated object and including an inner space preferably formed by a structure of a plurality of layers for receiving or mounting at least one functional unit including a wireless payment or data transaction unit having a secure element adapted for enabling or conducting a secure wireless payment or data transaction with an external matched or associated apparatus; and/or a health monitoring unit including at least one sensor and/or status monitor/controller for sensing, storing, and/or analysing a biometric data adapted and configured for health monitoring; and preferably the token smart ring is configured to be switchable between a health monitoring state and a secure data transaction state.

### TECHNICAL FIELD

The present disclosure relates to contactless secure transaction methods and systems such as for mobile payments or data transactions, and more particularly to a token smart ring for conducting wireless secure payment and/or data transaction and/or health monitoring.

### BACKGROUND ART

The smart mobile devices, such as smartwatches, adopted by the contactless payment systems utilizing the secure element generally comprise a contactless active payment unit or module needs to be powered by an embedded powerful battery thereof. However, quite a few people do not want to use any smartwatch and prefer traditional watches for various reasons. Moreover, it is desirable that the existing secure element for smart mobile devices could be also adapted for use in other kinds of contactless secure data transaction/communcation system apart from the contactless payment system.

### BRIEF SUMMARY OF INVENTION

The present disclosure relates to a battery-less active and passive hybrid device for secure wireless data transaction comprising a secure element adapted for storing at least one secure applet and at least one data segment for secure contactless data transaction and operatively connected with an active operation unit and a passive operation unit; and preferably the active operation unit is adapted to be connected removably with the secure element; and wherein the hybrid device is configured to be switchable between an active state in which the active operation unit is activated on demand to enable the hybrid device to function as an active device and adapted for provisioning one or more personalization information data, biometric data, health data, government related security data, hardware specific data, and/or a token of the device to the secure element; and a passive state in which the active operation unit is deactivated and the passive operation unit is activated/configured to enable the hybrid device to function as a passive device and adapted for conducting data verification and/or contactless data transaction operation via the secure element. As the hybrid device is battery-less and activated on demand, it can be incorporated into a traditional wearable device such as a ring or the like for provision of contactless payment capability.

The present disclosure relates to a token smart ring, preferably adapted for wireless payment and/or health monitoring, comprising a main portion adapted for engaging with an external associated object and including an inner space preferably formed by a structure of a plurality of layers for receiving or mounting at least one functional unit; wherein the at least one functional unit including:
a wireless payment or data transaction unit comprising a secure element adapted for enabling or conducting a secure wireless payment or data transaction with an external matched or associated apparatus; and/or
a health monitoring unit comprising at least one sensor and/or status monitor/controller for sensing, storing, and/or analysing a biometric data adapted and configured for health monitoring;
preferably the at least one sensor and/or status monitor/controller comprising a heart rate sensor for sensing a human heart rate; a temperature sensor for sensing ambient or surrounding temperature and/or a human body temperature; and/or an accelerometer sensor for measuring seismic activity, inclination, vibration, dynamic distance and speed of the token smart ring with or without the influence of gravity; and/or
a heart rate monitor for measuring and storing/recording heart rate in a real-time manner; and/or
a calories burned calculator for computing or estimating how many calories being burned during a predetermined routine daily human activity or a workout or exercise based on a predetermined activity duration including a day, a week, and/or a month; and/or
a pedometer or step counting monitor for measuring or computing a number of human steps and/or a human travel distance or a walk mileage; and/or for tracking or recording multiple metrics including a heart rate, asleep status, a stress status, a blood oxygen level, a calories burned value, and/or an activity duration; and/or
a sleep status monitor for computing or recording a sleep status data including a wake up time or a bed time based on a preset duration of sleep required, whereby facilitating a determination of an optimal sleep schedule based on a human unique situation including an age, awake up time and/or a bedtime; and preferably the sleep status data including a data obtained respectively in a Non-REM (rapid eye movement) sleep stages including awake, light, and deep sleep stages, and a REM (rapid eye movement) sleep stage of a sleep; and/or
a thermometer adapted for a body temperature measurement; and/or
a data storage unit adapted for storing and presenting a predetermined set of data including a heart rate data, a calorie data, a step counting data, a sleep duration data, and/or a body temperature data; and preferably the predetermined set of data is configured to be stored up to and/or valid through at least 14 days;
and preferably the token smart ring is configured to be switchable between a health monitoring state and a secure data transaction state; wherein in the health monitoring state the health monitoring unit is activated and configured for sensing, storing, and/or analysing the biometric data originated or came from an engageable portion of the external associated object for facilitating a human health monitoring and/or promoting operation; while in the secure data transaction state the health monitoring unit is deactivated and the wireless payment or data transaction unit and/or the secure element is activated to make the token smart ring to act as an active device adapted for provisioning one or more personalization information data, biometric data, health data, government related security data, hardware specific data, and/or a token of the smart ring to the secure element or a passive device adapted for conducting wireless secure payment, data verification and/or contactless data transaction operation via the secure element.

In some embodiments, wherein the main portion substantial comprised or made of a plurality of layers including three layers preferably of different materials, and preferably including an innermost layer substantially made of ceramic adapted for engaging the ring with the external associated object, an intermediate layer substantially made of stainless steel configured for mounting the at least one functional unit at the ring, and an outer layer substantially made of plastic adapted for coupling with the innermost layer to form the inner space for receiving the intermediate layer and the at least one functional unit.

In some other embodiments, wherein the wireless payment or data transaction unit is a battery-less active and passive hybrid device for secure wireless data transaction comprising a secure element adapted for storing at least one secure applet and at least one data segment for secure contactless data transaction and operatively connected with an active operation unit and a passive operation unit; and preferably the active operation unit is adapted to be connected removably with the secure element; and wherein the hybrid device is configured to be switchable between an active state in which the active operation unit is activated on demand to enable the hybrid device to function as an active device and adapted for provisioning one or more personalization information data, biometric data, health data, government related security data, hardware specific data, and/or a token of the device to the secure element; and a passive state in which the active operation unit is deactivated and the passive operation unit is activated/configured to enable the hybrid device to function as a passive device and adapted for conducting data verification and/or contactless data transaction operation via the secure element.

In several embodiments of the token smart ring according to the present disclosure, wherein it is configured to have a water resistant level of IPX7; and/or it further comprises a power unit adapted for powering the token smart ring to conduct a desired operation for at least 3 days and lasting for up to 6 years depending on usage of the smart ring or frequency of the desired operation, and preferably comprises a data transfer interface and/or a power charging interface for engaging with an external associated transfer device for conducting a data transfer and/or a power transfer therebetween.

In other embodiments, wherein the data transfer interface and/or the power charging interface comprises a female type connector with a plurality of female contacts hidden from view for receiving or removably coupling with a male type connector of the external associated transfer device with an outer dimension or an outer profile in line with an inner profile of the main portion for forming a removable tight fit with the token smart ring for conducting data or power transfer therewith.

In further embodiments, wherein the female type connector comprises a rounded edge female contact substantially in form a circular hole; and the male type connector comprises a rounded edge male contact substantially in form a circular pin being movable along or in a radial direction and insertable into or removable from the circular hole when engaging or disengaging with the female type connector; and/or the external associated transfer device has an round outer dimension being insertable into or removable from an inner cavity of the main portion for conducting or switching off a data or power transfer therewith.

In other embodiments, wherein the female type connector and/or the external associated transfer device comprise a visual and/or audio indicator for indicating a working state or status and/or a working result of a data or power transfer operation carried out therebetween.

In additional embodiments, further comprising a WIFI, BT including BLE, and/or NFC enabled communication unit for conducting wireless secure data transaction and/or wireless secure payment with the external matched or associated apparatus; and preferably the communication unit is configured for conducting wireless secure payment with an external matched or associated POS terminal.

In some other embodiments, wherein the associated transfer device comprises a first cylindrical part substantially in the form of a cylinder, while one end of its opposite ends is removably engaged or integrated with an interface unit for power and/or data transfer whereby enabling a provisioning operation and/or a charging operation upon coupling with the token smart ring; wherein another end of the opposite ends is removably engaged or integrated with a second cylindrical part substantially also in the form of a cylinder with a diameter small than that of the first cylindrical part and adapted for insertion into or coupling with the token smart ring whereby provisioning or charging it once engaged therewith; wherein the first cylindrical part acts as an alignment member and/or a stopper for the token smart ring during its coupling with the second cylindrical part.

In some embodiments, wherein it has a width of 10 mm +/−0.5 mm or less, a thickness of 4.5 mm +/−0.5 mm or less, and/or a weight of 6-8 grams or less.

The token smart ring provided by the present disclosure is simple in structure, reasonable in design, high in comfort and low in cost, such that it enables the provision of a versatile wearable device for secure wireless payment, secure wireless data transaction/communication, and/or health monitoring.

### BRIEF DESCRIPTION OF DRAWINGS

The present disclosure will be described in details below with reference to the accompanying drawings, in which:

FIG. 1 is a block diagram of a battery-less active and passive hybrid device according to a preferred embodiment of the present disclosure;

FIG. 2 is a block diagram of a battery-less active and passive hybrid device according to another preferred embodiment of the present disclosure;

FIG. 3 is a flow chart of a method for providing contactless payment capabilities to a traditional wearable device by a battery-less active and passive hybrid device according to a further preferred embodiment of the present disclosure;

FIG. 4 is a block diagram of a battery-less active and passive hybrid device according to another preferred embodiment of the present disclosure;

FIG. 5 is a block diagram of a battery-less active and passive hybrid device according to yet still another preferred embodiment of the present disclosure;

FIG. 6 is a schematic view of a watch band adopting the battery-less active and passive hybrid device according to one preferred embodiment of the present disclosure;

FIG. 7 is a schematic view of a watch band adopting the battery-less active and passive hybrid device according to another preferred embodiment of the present disclosure;

FIG. 8 is a block diagram of a battery-less active and passive hybrid device according to another preferred embodiment of the present disclosure;

FIG. 9 is a block diagram of a battery-less active and passive hybrid device according to yet still another preferred embodiment of the present disclosure;

FIG. 10a is a schematic view of an universal passive provisioning unit adopted by the battery-less active and passive hybrid device according to one preferred embodiment of the present disclosure;

FIG. 10b is a schematic view of a clip type universal passive provisioning unit adopted by the battery-less active and passive hybrid device according to another preferred embodiment of the present disclosure;

FIGS. 11a and 11b are schematic views of a token smart ring and its accessary adapted for use with the universal passive provisioning unit adopted by the battery-less active and passive hybrid device according to one preferred embodiment of the present disclosure; and

FIG. 12 is a schematic sectional view of the token smart ring adapted for use with the universal passive provisioning unit adopted by the battery-less active and passive hybrid device according to another preferred embodiment of the present disclosure.

### DETAILED DESCRIPTION OF INVENTION

The present disclosure will now be described in further details with reference to the accompanying drawings and embodiments, so as to make the objects, technical solutions and advantages of the present disclosure more apparent.

The present disclosure relates to devices and methods for use with EMVCo payment applets provisioning processes on secure element residing on a battery-less and bendable active board environment. The respective terms “EMVCo” or “EMV” means a consortium of Europay, MasterCard, Visa and others, “SE” means a secure element, “NFC” means Near Field Communications or relevant protocols, “BLE” means Bluetooth low energy or the like, and “Payment Applets” means contactless applications residing in SE.

Further, the term “Bendable Active Board” means a platform of bendable nature that consists of a SE for storing secure payment applets and tokens; a Bluetooth module for communications with paired devices; an NFC inductance antenna to receive power from NFC point of sale terminal in turn powering the SE; a wireless interface to receive power from an external source to enable the Bluetooth module on demand.

The respective terms “API” means application programming interface; “PAN” means a payment card primary account number; “Token” means a surrogate replacing the PAN, “Token services” means API offerings from payment network schemes not limited to Visa and Mastercard, where the primary functions are to exchange payment card PANs with surrogates as well as the management of such payment accounts; “Token Band” means an active or passive wearable device or strap/band capable of storing payment token; ‘Token Dock” means a standalone power source docking system capable of transferring power wirelessly to the to Token Band; “Basic Wearable” means wearable that do not support loading of third party applications; and “Smart Wearable” means wearable that can support loading of third party applications.

In addition, the term “passive provisioning unit”, “passive data provisioning unit”, or the term “active operation unit” used herein means a passive or battery-less device or component capable of enabling an apparatus comprising a secure element, with which it operatively connected, coupled, or cooperated, to function as an active device on demand and adapted for provisioning one or more personalization information data, biometric data, health data, government related security data, hardware specific data, and/or a token of the apparatus/device to the secure element in electric and/or signal connection therewith; and the term “passive operation unit” means a passive or battery-less device or component capable of enabling the apparatus with which it operatively connected, coupled, or cooperated to function as a passive device on demand and adapted for conducting data verification and/or contactless data transaction operation, such as payment transaction operation, via the secure element.

Referring to FIG. 1, a block diagram of a battery-less active and passive hybrid device according to a preferred embodiment of the present disclosure is illustrated, according to which the hybrid device for secure wireless payment comprises a secure element 20 adapted for storing at least one secure payment applet and at least one token for secure contactless payment and operatively connected with a passive data provisioning unit/an active operation unit 10 and a passive operation unit 30.

The hybrid device is configured to be switchable between an active state and a passive state in response to the working requirements and conditions for various operations as required. In the active state, the active operation unit 10 is activated and powered preferably wirelessly on demand, e.g, by an external power source or initiator/activator, to enable the hybrid device to function as an active device, and it is adapted for various operations requiring a relatively high and stable power consumption, among other, the operations for provisioning a personalization information of a payment card and/or a payment token of the device to the secure element. In this regard, the active operation unit enables the hybrid device to support installing and loading of third party applications by the end user.

In the passive state, the active operation unit is deactivated, and/or decoupled with external initiator, and the passive operation unit is activated to enable the hybrid device to function as a passive device and adapted for various operations requiring a relatively low or transient power consumption, among other, conducting payment token verification and/or contactless payment transaction operation via the secure element, wherein the low or transient operating power could be drawn from a predetermined matching device adapted for working with a passive device.

In some embodiments such as the embodiment as shown in the FIG. 1, while in the active state the active operation unit 10 is activated by operatively coupled with and powered wirelessly by a wireless power transfer unit 60 for conducting wireless data communication with a provisioning unit, such as a dedicated machine, or a computer, a smart or mobile device comes with a dedicated module for provisioning data to the secure element 20, whereby enabling the provisioning of the personalization information of the payment card and/or the payment token of the device to the secure element from the provisioning unit.

In some embodiment such as the embodiment as shown in the FIG. 1, while in the passive state the passive operation unit 30 is activated by operatively coupled with and wirelessly powered by a payment transaction unit 50, such as a dedicated machine terminal, or a computer, a smart or mobile device comes with a dedicated module for contactless payment transaction with the secure element 20, whereby enabling the conducting of payment token verification and/or contactless payment transaction operation with the payment transaction unit via the secure element.

In some embodiments, the active operation unit comprises a first wireless communication unit adapted for coupling and conducting wireless data communication with the personalization information and payment token provisioning unit. The first wireless communication unit is operatively coupled with a wireless power receiver unit and/or a transient power storage unit, preferably a supercap, adapted for receiving and/or storing power from the coupled wireless power transfer unit, so as to enable the completion of routine and specific operations of the hybrid device requiring a relatively high and stable operating power.

In some embodiments, the first wireless communication unit is aWIFI, BLE, and/or NFC enabled communication unit.

In some embodiments, the passive operation unit comprises a second wireless communication unit, adapted for coupling and conducting wireless data communication with the payment transaction unit and operatively coupled with an antenna unit for receiving its operating power and payment transaction data and signals from the coupled payment transaction unit.

In some embodiments, the second wireless communication unit is a NFC enabled communication unit acting as a NFC passive target unit configured to draw its operating power from the payment transaction unit acting as a NFC initiator unit.

In other embodiments, the first and second wireless communication unit are both or the same NFC enabled communication unit, namely the first wireless communication unit and the provisioning unit are both active mode NFC communication unit, as NFC devices can work in peer-to-peer mode, which enables two active mode NFC-enabled devices to communicate with each other to exchange information in an adhoc fashion, wherein one of the devices will deactivate its RF field while it is waiting for data.

In some embodiments, the antenna unit comprises an antenna of a dimension of 10×24mm. The dimension is desirable for conducting stable and reliable data communication with the payment transaction unit without the need of incorporating an antenna booster to ensure proper operations of the passive operation unit, as could be found in some prior art devices using a relatively small or miniature antenna. If the dimension of the antenna is relative large, then the hybrid device could not entirely fit into some traditional wearable devices, such as a traditional watch and jewellery.

In some embodiments, the secure element, the active operation unit, and the passive operation unit are configured to be operatively coupled with each other and mounted on a bendable board adapted for easy mounting on a non-planar or a curved surface or being readily insertable or embedded into a curved accommodating space.

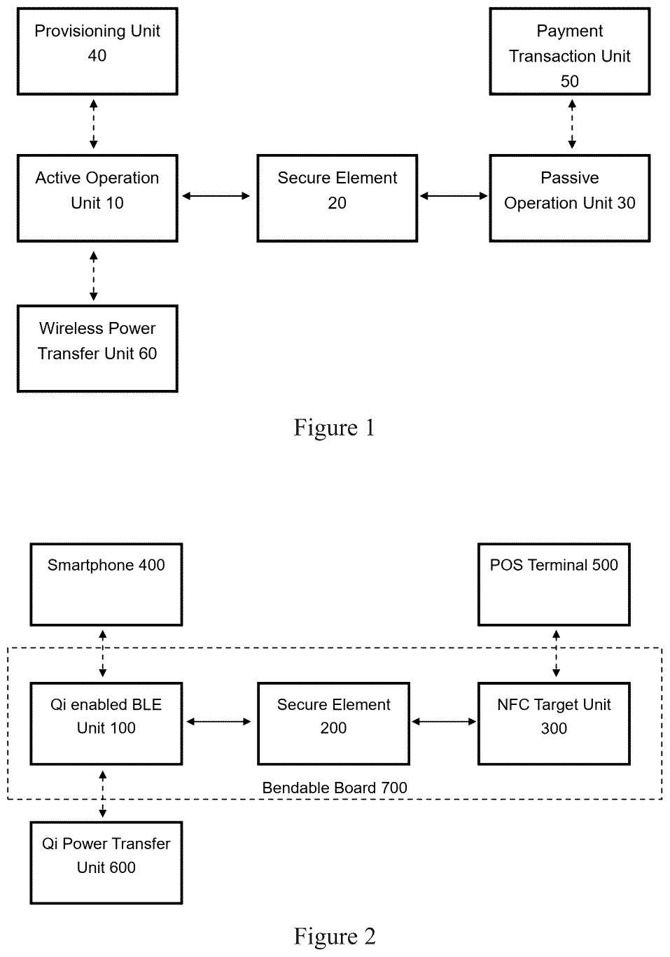

Now referring to FIG. 2, a block diagram of a battery-less active and passive hybrid device according to another preferred embodiment of the present disclosure is illustrated, according to which the hybrid device for secure wireless payment comprises a secure element 200 adapted for storing at least one secure payment applet and at least one token for secure contactless payment and operatively connected with a Qi and BLE enabled active operation unit and a NFC enabled passive operation unit.

In some embodiments such as the embodiment as shown in the FIG. 2, the active operation unit is a Qi enabled BLE unit 100 comprising a Qi standard power receiver and the wireless power transfer unit is a Qi enabled power transfer unit 600 comprising a Qi standard power transmitter for providing wirelessly the power to the BLE unit for conducting wireless data communication with a provisioning unit, namely a smartphone 400 as shown in the FIG. 2. The passive operation unit is a NFC passive target unit 300 operatively coupled with and wirelessly powered by a payment transaction unit, namely a POS terminal 500, and configured to draw its operating power from the payment transaction unit acting as a NFC initiator unit for conducting payment token verification and/or contactless payment transaction operation with the payment transaction unit via the secure element 200.

Further, in the hybrid device as shown in FIG. 2, the secure element 200, the Qi enabled BLE unit 100, and the NFC passive target unit 300 are operatively coupled with each other and mounted on a bendable board 700 adapted for easy mounting on a non-planar or a curved surface and/or being readily insertable or embedded into a curved accommodating space.

In some embodiments, the hybrid device is mounted at and preferably embedded into a band or buckle of a traditional watch to replace the original band or buckle of the watch of a user for conducting contactless payment transactions and other applicable operations, such that the user could wear and use the watch in a traditional way while benefit from the contactless payment capabilities provided by the new and replaceable part of the watch.

Traditional watches are composed of mostly mechanical components and metal materials for the casing attached to a band or wrist strap. The wrist strap also can be produced in a variety of materials including leather, plastic, metals etc. Adding smart features such as payment applets on this segment of wearable under normal circumstances require a battery source to enable communications with external devices such as a smart phone.

The challenges or issues with adding an integrated battery source to a traditional wearable product are as follows:
design and aesthetic issue, wherein the battery is rigid and the placement can take up space adding unnecessary thickness impacting aesthetic and designs;
battery recharging issue, wherein the recurring use of a rechargeable battery requires added components and interface such as cable attachment to the device impacting aesthetics and productions costs of the final product;
battery replacement issue, wherein all battery has a limited lifespan which needs replacement, whether or not the battery is rechargeable; and
water resistant issue, wherein enclosure of battery and recharging cable interface can limit the water resistant ability of the overall device.

Accordingly, the technical solutions set forth by the present disclosure could apparently solve the foregoing technical problems for providing power to traditional and basic wearable product (including but not limited to, timepiece and jewellery) on demand while removing the dependency and need of an integrated battery. The present disclosure combines a bendable board enabling a unique payment token provisioning sequence to add NFC payment capabilities on traditional and basic wearable products without the need of an integrated battery source. The ability to decouple the battery from the wearable while having the ability to enable communications with smart devices through Bluetooth or similar technology is critical in overcoming all of foregoing challenges or issues in the prior art.

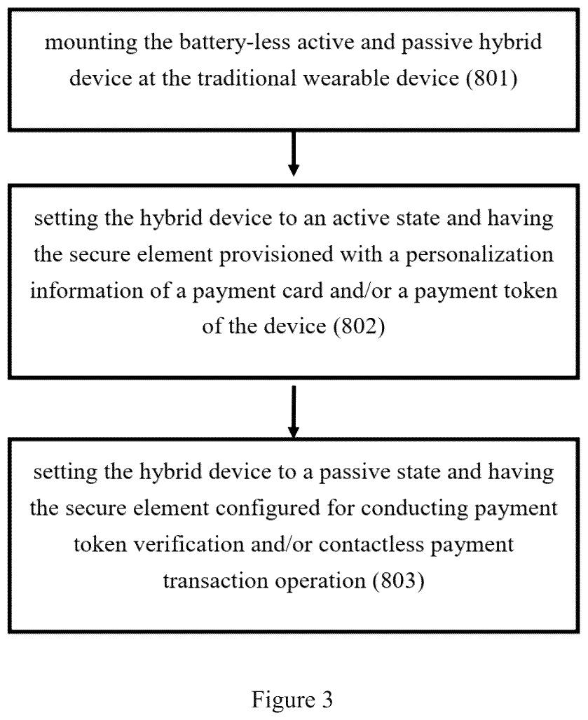

Referring to FIG. 3, which illustrates a flow chart for a method for providing contactless payment capabilities to a traditional wearable device by a battery-less active and passive hybrid device for secure wireless payment comprising a secure element adapted for storing at least one secure payment applet and at least one token for secure contactless payment and operatively connected with a passive data provisioning unit/an active operation unit and a passive operation unit, comprising steps of:
mounting/embedding the battery-less active and passive hybrid device at/into the traditional wearable device (801);
switching/setting the hybrid device to an active state and having the secure element provisioned with a personalization information of a payment card and/or a payment token of the device (802); and switching/setting the hybrid device to a passive state and having the secure element configured for conducting payment token verification and/or contactless payment transaction operation (803).

In some embodiments, the foregoing method comprises one or more of the following optional steps of:
incorporating one or more biometric sensors (as will be described below in further details) with the hybrid device before mouting it into the i traditional wearable device;
pairing up the hybrid device and and a mobile device via BLE for receiving one or more additional personalization information of one or more payment cards from the mobile device for conducting one or more additional provisioning operations with the secure element; and/or
activating a biometric sensor incorporated with the hybrid device for conducting secondary authentication for conducting secure element provisioning process, payment token verification, and/or contactless payment transaction operation.

In some embodiments, the foregoing method comprises the steps of:
mounting/embedding the battery-less active and passive hybrid device at/into the traditional wearable device;
switching/setting the hybrid device to an active state by having the active operation unit activated wirelessly, or by an engageable and decoupleable or removably/decoupleably
connected external power transfer unit, on demand to enable the hybrid device to function as an active device and the secure element provisioned with a personalization information of a payment card and/or a payment token of the device; and
switching/setting the hybrid device to a passive state by having the active operation unit deactivated wirelessly, or by disengaging/decoupleabling with the external power transfer
unit, on demand and the passive operation unit activated/configured to enable the hybrid device to function as a passive device and the secure element configured for conducting payment token verification and/or contactless payment transaction operation.

According to another aspect of the present disclosure, which provides a provisioning process flow for a battery-less active and passive hybrid device for secure wireless payment comprising a secure element adapted for storing at least one secure payment applet and at least one token for secure contactless payment as follows:

Step 1: Place the token band or the hybrid device with the secure element on top of an activated or powered token dock to receive wirelessly power from the token dock to power its BLE unit.

Step 2: Start a mobile application on a smartphone and complete the standard BLE pairing with the token band to initiate token services provisioning request with payment networks.

Step 3: The mobile application subsequently communicates with the SE over global platform standards and completes the personalization of the payment token securely on the SE.

Step 4: Remove the token band from the token dock and place the nonpowered passive token band over an NFC enabled point of sales terminal, which exerts power to an antenna on the token band to communicate with the payment applet and complete a NFC contactless payment transaction with the terminal.

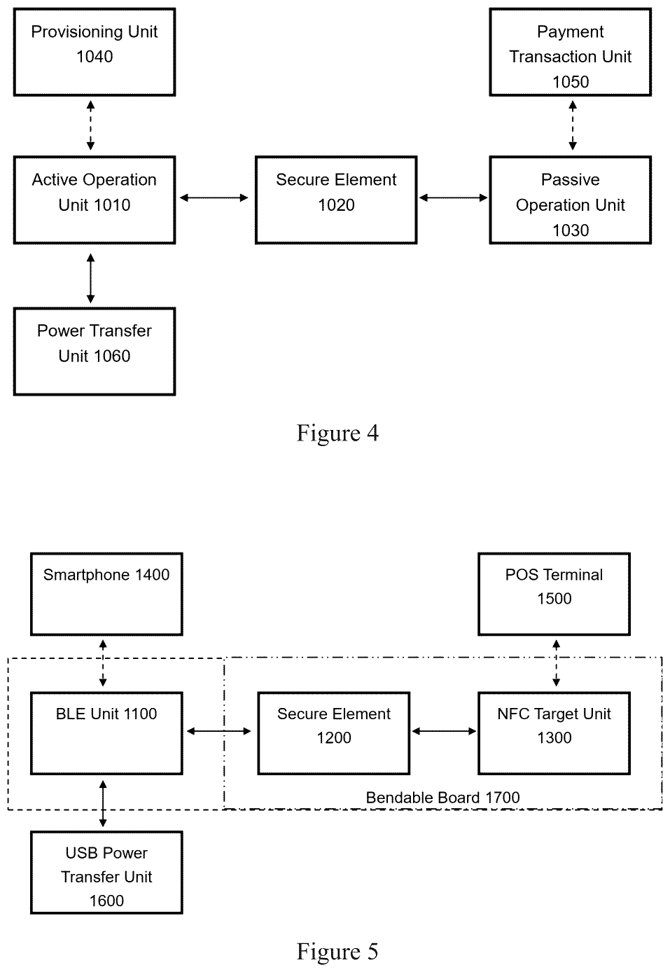

Referring to FIG. 4, a block diagram of a battery-less active and passive hybrid device according to a preferred embodiment of the present disclosure is illustrated, according to which the hybrid device for secure wireless payment comprises a secure element 1020 adapted for storing at least one secure payment applet and at least one token for secure contactless payment and operatively connected with a passive data provisioning unit/an active operation unit 1010 and a passive operation unit 1030. The present embodiment and the embodiments as shown in FIG. 1 and FIG. 2 differ in that the active operation unit is preferably adapted to be engageable and decoupleable or connected removably/decoupleably with the secure element. For example, the active operation unit could be integrated with the power transfer unit or an interface for power transfer (such as an USB interface for receiving power from an external USB power source/socket/cable) and/or not to be mounted together nor connected permanently with the secure element and the passive operation unit on the hybrid device/the bendable board.

The hybrid device is configured to be switchable between an active state and a passive state in response to the working requirements and conditions for various operations as required. In the active state, the active operation unit 1010 is activated and powered on demand, e.g, by an external power source, to enable the hybrid device to function as an active device, and it is adapted for various operations requiring a relatively high and stable power consumption, among other, the operations for provisioning a personalization information of a payment card and/or a payment token of the device to the secure element. In this regard, the active operation unit enables the hybrid device to support installing and loading of third party applications to the secure element by the end user.

In the passive state, the active operation unit is deactivated, and/or decoupled with external power source, and the passive operation unit is activated/configured to enable the hybrid device to function as a passive device and adapted for various operations requiring a relatively low or transient power consumption, among other, conducting payment token verification and/or contactless payment transaction operation via the secure element, wherein the low or transient operating power could be drawn from a predetermined matching device adapted for working with a passive device.

In some embodiments such as the embodiment as shown in the FIG. 4, while in the active state the active operation unit 1010 is activated by operatively coupled with and powered by a power transfer unit 1060 for conducting wireless data communication with a provisioning unit, such as a dedicated machine, or a computer, a smart or mobile device comes with a dedicated module for provisioning data to the secure element 1020, whereby enabling the provisioning of the personalization information of the payment card, biometric data, health data, government related security data, hardware specific data, and/or the payment token of the device to the secure element from the provisioning unit.

In some embodiment such as the embodiment as shown in the FIG. 4, while in the passive state the passive operation unit 1030 is activated by operatively coupled with and wirelessly powered by a payment transaction unit 1050, such as a dedicated machine terminal, or a computer, a smart or mobile device comes with a dedicated module for contactless payment transaction with the secure element 1020, whereby enabling the conducting of payment token verification and/or contactless payment transaction operation with the payment transaction unit via the secure element.

In some embodiments, the active operation unit comprises a first wireless communication unit adapted for coupling and conducting wireless data communication with the personalization information and payment token provisioning unit. The first wireless communication unit is operatively coupled with a power receiver unit and/or a transient power storage unit, preferably a supercap, adapted for receiving and/or storing power from the coupled power transfer unit, so as to enable the completion of routine and specific operations of the hybrid device requiring a relatively high and stable operating power.

In some embodiments, the first wireless communication unit is a WIFI, BT, and/or NFC enabled communication unit, and preferably a BLE unit with a plurality of contacts (and preferably, 2 or 4 contacts), preferably waterproof and/or exposed contacts.

In some embodiments, the passive operation unit comprises a second wireless communication unit, adapted for coupling and conducting wireless data communication with the payment transaction unit and operatively coupled with an antenna unit for receiving its operating power and payment transaction data and signals from the coupled payment transaction unit.

In some embodiments, the second wireless communication unit is a NFC enabled communication unit acting as a NFC passive target unit configured to draw its operating power from the payment transaction unit acting as a NFC initiator unit.

In other embodiments, the first and second wireless communication unit are both or the same NFC enabled communication unit, namely the first wireless communication unit and the provisioning unit are both active mode NFC communication unit, as NFC devices can work in peer-to-peer mode, which enables two active mode NFC-enabled devices to communicate with each other to exchange information in an adhoc fashion, wherein one of the devices will deactivate its RF field while it is waiting for data.

In some embodiments, the antenna unit comprises an antenna of a preferred dimension of 10×30mm, and more preferably 10×24mm. The dimension is desirable for conducting stable and reliable data communication with the payment transaction unit without the need of incorporating an antenna booster to ensure proper operations of the passive operation unit, as could be found in some prior art devices using a relatively small or miniature antenna. If the dimension of the antenna is relative large, then the hybrid device might not be entirely fitted into some traditional wearable devices, such as a traditional watch and jewellery worn on wrist, waist, or neck, for example.

In some embodiments, the secure element, the active operation unit, and the passive operation unit are configured to be operatively coupled with each other and mounted on a bendable board adapted for easy mounting on a non-planar or a curved surface or being readily insertable or embedded into a curved accommodating space; or the active operation unit is adapted to be an external standalone unit and removably connected with the secure element and/or integrated with the power transfer unit rather than mounted on the bendable board.

Now referring to FIG. 5, a block diagram of a battery-less active and passive hybrid device according to another preferred embodiment of the present disclosure is illustrated, according to which the hybrid device for secure wireless payment comprises a secure element 1200 adapted for storing at least one secure payment applet and at least one token for secure contactless payment and operatively connected with a BLE enabled passive data provisioning unit/active operation unit and a NFC enabled passive operation unit.

In some embodiments such as the embodiment as shown in the FIG. 5, the active operation unit is a BLE unit 1100 comprising a plurality of contacts for receiving power from removably coupled power transmitter and the power transfer unit is a USB power transfer unit 1600 comprising a USB power transmitter, preferably equipped with connection members, such as connection pins or hooks, and/or fixing or locking members for making contact and/or interlocking with the contacts of the BLE unit, whereby providing the power to the BLE unit for conducting wireless data communication with a provisioning unit, namely a smartphone 1400 as shown in the FIG. 5. The passive operation unit is a NFC passive target unit 1300 operatively coupled with and wirelessly powered by a payment transaction unit, namely a POS terminal 1500, and configured to draw its operating power from the payment transaction unit acting as a NFC initiator unit for conducting payment token verification and/or contactless payment transaction operation with the payment transaction unit via the secure element 1200.

Further, in the hybrid device as shown in FIG. 5, the secure element 1200, the BLE unit 1100, and the NFC passive target unit 1300 are operatively coupled with each other and mounted on a bendable board 1700 adapted for easy mounting on a non-planar or a curved surface and/or being readily insertable or embedded into a curved accommodating space. Alternatively, the BLE unit is adapted to be removably connected with the secure element without mounting fixedly on the bendable board; and/or the BLE unit might be integrated with the USB power transfer unit 1600, as will be described in further details below.

In some embodiments, the hybrid device is mounted at and preferably embedded into a band or buckle, preferably made of a non-metal material such as leather or plastic or the like to reduce the interference thereof, of a traditional watch to replace the original band or buckle of the watch of a user for conducting contactless payment transactions and other applicable operations, such that the user could wear and use the watch in a traditional way while benefit from the contactless payment capabilities provided by the new and replaceable part of the watch.

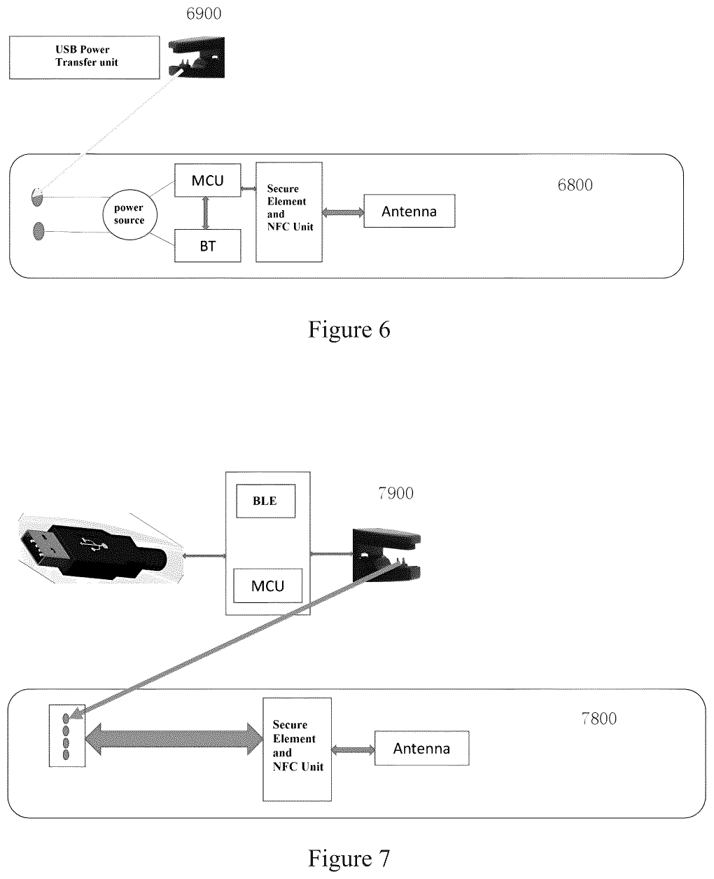

Referring to FIG. 6, a schematic view of a watch band 6800 adopting the battery-less active and passive hybrid device according to one preferred embodiment of the present disclosure is illustrated, wherein the power transfer unit is a USB power transfer unit 6900 substantially in the form of a clip/clamp or a forepart of clothes-pin. The USB power transfer unit comprises an upper part and a lower part on which a plurality of connection pins or hooks extended from the base of the lower part along a longitudinal axis are mounted firmly and adapted to engage with the respective contacts arranged in the watch band. The USB power transfer unit might further comprise at least one fixing or locking members (not shown) for making contact and/or interlocking with the contacts of the BLE unit. The power received through the contacts are used to power or activate directly the active operation unit, or activate an optional internal power source/accumulator (such as a supercap in electric connection with the contacts for receiving and storing power from the coupled USB power transfer unit) of the hybrid device embedded in the watch band for powering the active operation unit, comprising a MCU and BT/BLE unit operatively connected with the secure element and NFC unit. While in the passive state, the antenna of the passive unit will draws its operating power wirelessly from external payment transaction unit for powering the passive operation unit comprising the NFC unit operatively connected with the secure element and the antenna.

Now referring to FIG. 7, a schematic view of a watch band 7800 adopting the battery-less active and passive hybrid device according to another preferred embodiment of the present disclosure is illustrated, wherein the power transfer unit is a USB power transfer unit 7900 also substantially in the form of a clip/clamp or a forepart of clothes-pin and integrated with the active operation unit comprising the BLE unit and the MCU. In this regard, the active operation unit is removably connected with the secure element and only the secure element and the passive operation unit will be mounted on the bendable board and embedded into the watch band, and such configuration will simplify substantially the layout and design of the hidden or concealed part of the hybrid device being mounted and embedded into the wearable device, such as the watch band.

Similarly, the USB power transfer unit comprises an upper part and a lower part on which a plurality of connection pins or hooks extended from the base of the lower part along a longitudinal axis are mounted firmly and adapted to engage with the respective contacts arranged in the watch band and/or on the bendable board on which the secure element and passive operation unit are mounted. The USB power transfer unit might further comprise at least one fixing or locking members (not shown) for making contact and/or interlocking with the contacts operatively connected with the secure element and the NFC unit. The coupling between the connection pins and the contacts is adapted for provisioning personalization information of a payment card and/or a payment token of the device to the secure element via the external active operation unit. The contacts are adapted for enabling the power transmission and data communication between the external action operation unit and the secure element. In this embodiment, there are four contacts such that data and power signal could be transferred concurrently to facilitate the operation of the active operation unit and the secure element. While in the passive state, the antenna will draws its operating power wirelessly from external payment transaction unit for powering the passive operation unit comprising the NFC unit operatively connected with the secure element and the antenna.

In some embodiments, the watch band and/or the bendable board comprise one or more optional second contacts adapted for provision of additional functions. For example, one of the optional second contacts is a reset contact to enable a warm or cold reboot/reset of the operating system/the kernel of the secure element when necessary. In this regard, the power transfer unit also comprises one or more optional second connection pins or hooks adapted for making electric connection with the respective second contacts for implementation of the relevant additional functions.

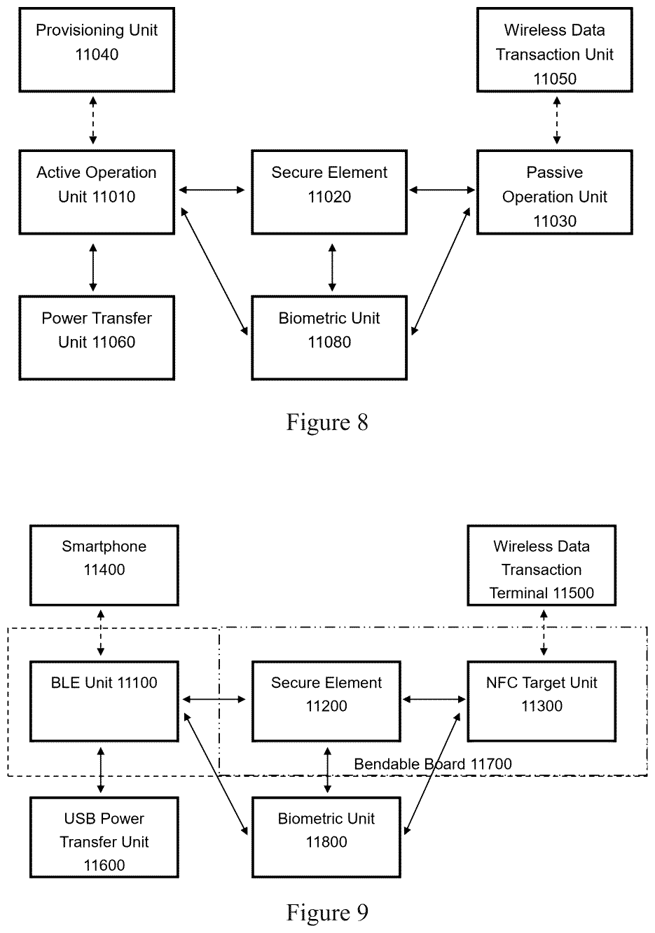

Referring to FIG. 8, a block diagram of a battery-less active and passive hybrid device according to a preferred embodiment of the present disclosure is illustrated, according to which the hybrid device adapted for various secure applications/operations/communication comprises a secure element 11020 adapted for storing at least one secure applet for conducting various secure applications/operations and at least one data segment/memory for secure contactless data transaction/exchange and operatively connected with an active operation unit 11010 and a passive operation unit 11030. The present embodiment is similar to the one as shown in FIG. 4 in structural, operational, and functional aspects, while the present embodiment and the embodiments as shown in FIGS. 1-2 and FIGS. 4-5 differ basically in that it further comprise a biometric unit 11080 for secondary or biometric authentication of user identity comprising one or more biometric readers/sensors capable of reading, writing and/or processing biometric data related to one or more of the following items/entries: fingerprint, palm/finger vein pattern, voice pattern, face recognition, DNA, palm print, hand geometry, iris recognition, and/or retina. The biometric unit 11080 is preferably a passive device and operatively connected with the secure element 11020, the active operation unit 11010, and the passive operation unit 11030 as shown in the figure. Definitely and apparently, the biometric unit could be also incorporated by and for use with all of the above embodiments in a similar manner.

Further, the hybrid device of the present embodiment is adapted not only for wireless payment but also for wireless data transaction/communication for use in various secure applications/operations as the secure element operated in active state could be provisioned with not only information of payment card and token for payment transaction, but also biometric data, health data, hardware specific data, and/or Government related security data and the like. In this regard, the hybrid device could be also adapted at least for use with various access control systems, intelligent remote key systems for automobile, medical systems, or the like.

In some embodiments, the hybrid device or the secure element operated in active state is provisioned with personalization information and/or biometric data of authorized user/personnel and a secure applet configured for use with an access control system via the provisioning unit 11040 and active operation unit 11010. In operation, the hybrid device operated in passive state plays the role of an access card or ID badge of the access control system, and the passive operation unit 11030 draws its operating power from a card reader or a dedicated wireless data transaction unit 11050 playing the role of the card reader of the access control system, such that the secure applet is activated to communicate wirelessly with the access control system for verification of user identity whereby opening respective doors controlled by the access control system if the verification result is positive. If a secondary authentication or biometric data is required by the access control system, the secure element or the secure applet operated in passive state receives and/or processes the biometric data of user provided by the biometric unit and forward wirelessly the original or processed biometric data to the access control system to ensure the security thereof.

In some embodiments, the hybrid device or the secure element operated in active state is provisioned with hardware specific data (such as intelligent key data of intelligent key system for one or more automobiles for activation and proper operations of the automobiles) and/or biometric data of authorized person/driver and a secure applet configured for use with the respective intelligent key systems for automobile via the provisioning unit 11040 and active operation unit 11010. In operation, the hybrid device operated in passive state plays the role of an intelligent key or remote controller of the intelligent key system embedded or installed in the automobile, and the passive operation unit 11030 draws its operating power from a key reader or a dedicated wireless data transaction unit 11050 playing the role of the key reader of the intelligent key system of the automobile, such that the secure applet is activated to communicate wirelessly with the key system of the automobile for verification of driver identity whereby activating and operating respective parts controlled by the intelligent key system if the verification result is positive. If a secondary authentication or biometric data is required by the intelligent key system, the secure element or the secure applet operated in passive state receives and/or processes the biometric data of user provided by the biometric unit and forward wirelessly the original or processed biometric data to the intelligent key system to ensure the security and proper operation thereof.

In some embodiments, the hybrid device or the secure element operated in active state is provisioned with health data and/or biometric data of a participant/patient registered with an intelligent medical system and a secure applet configured for use with the medical system via the provisioning unit 11040 and active operation unit 11010. The health data comprises physical condition data, medical history data, historical prescription data of previous treatments or diagnoses, or the like. In operation, the hybrid device operated in passive state plays the role of an intelligent medical card or a smart card for personal identification for the medical system, and the passive operation unit 11030 draws its operating power from a card reader or a dedicated wireless data transaction unit 11050 playing the role of the card reader of the intelligent medical system, such that the secure applet is activated to communicate wirelessly with the intelligent medical system for verification of patient identity whereby enabling the provision and accessing of the medical history or the like by medical staffs of the intelligent medical system if the verification result is positive. If a secondary authentication or biometric data is required by the intelligent medical system, the secure element or the secure applet operated in passive state receives and/or processes the biometric data of the participant/patient provided by the biometric unit and forward wirelessly the original or processed biometric data to the intelligent medical system to ensure the security and proper operation thereof.

In some embodiments, the hybrid device or the secure element operated in active state is provisioned with government related security data and/or biometric data of a citizen or a resident governed by the government and a secure applet configured for use with an intelligent system or a smart city program provided by the government via the provisioning unit 11040 and active operation unit 11010. The government related security data comprises ID card or passport data, immigration or travel record data, contact information data, driving licence data, or the like. In operation, the hybrid device operated in passive state plays the role of an smart ID card for personal identification required by various intelligent systems or services provided by the government, and the passive operation unit 11030 draws its operating power from a card reader or a dedicated wireless data transaction unit 11050 playing the role of the card reader, such that the secure applet is activated to communicate wirelessly with the intelligent system for verification of personal identity whereby enabling the employment and accessing of various intelligent systems or services provided by the government if the verification result is positive. If a secondary authentication or biometric data is required by the intelligent system or services, the secure element or the secure applet operated in passive state receives and/or processes the biometric data of the participant/patient provided by the biometric unit and forward wirelessly the original or processed biometric data to the intelligent system to ensure the security and proper operation thereof.

The hybrid device is configured to be switchable between an active state and a passive state in response to the working requirements and conditions for various operations as required. In the active state, the active operation unit 11010 is activated and powered on demand, e.g, by an external power source, to enable the hybrid device to function as an active device, and it is adapted for various operations requiring a relatively high and stable power consumption, among other, the operations for provisioning one or more personalization information data, biometric data, health data, government related security data, hardware specific identification data and/or a token of the device to the secure element. In this regard, the active operation unit enables the hybrid device to support installing and loading of third party applications and application-specific data to the secure element by the end user.

In the passive state, the active operation unit is deactivated, and/or decoupled with external power source, and the passive operation unit is activated/configured to enable the hybrid device to function as a passive device and adapted for various operations requiring a relatively low or transient power consumption, among other, conducting data verification and/or contactless data transaction operation via the secure element, wherein the low or transient operating power could be drawn from a predetermined matching device adapted for working with a passive device.

In some embodiments such as the embodiment as shown in the FIG. 8, while in the active state the active operation unit 11010 is activated by operatively coupled with and powered by a power transfer unit 11060 for conducting wireless data communication with a provisioning unit, such as a dedicated machine, or a computer, a smart or mobile device comes with a dedicated module for provisioning data to the secure element 11020, whereby enabling the provisioning of the personalization information or the like and/or the token of the device to the secure element from the provisioning unit.

In some embodiment such as the embodiment as shown in the FIG. 8, while in the passive state the passive operation unit 11030 is activated by operatively coupled with and wirelessly powered by a data transaction unit 11050, such as a dedicated machine terminal, or a computer, a smart or mobile device comes with a dedicated module for contactless payment transaction with the secure element 11020, whereby enabling the conducting of data verification and/or contactless data transaction operation with the payment transaction unit via the secure element.

Now referring to FIG. 9, a block diagram of a battery-less active and passive hybrid device according to another preferred embodiment of the present disclosure is illustrated, according to which the hybrid device adapted for various secure applications/operations/communication comprises a secure element 11200 adapted for storing at least one secure applet for conducting various secure applications/operations and at least one data segment/memory for secure contactless data transaction/exchange and operatively connected with a BLE enabled active operation unit and a NFC enabled passive operation unit. The present embodiment is similar to the one as shown in FIG. 5 in structural, operational, and functional aspects, while the present embodiment and the embodiments as shown in FIGS. 1-2 and FIGS. 4-5 differ basically in that it further comprise a biometric unit 11800 for secondary or biometric authentication of user identity comprising one or more biometric readers/sensors capable of reading, writing and processing biometric data related to vein pattern, fingerprints, hand geometry, DNA, voice pattern, iris pattern, signature dynamics and/or face detection. The biometric unit 11800 is operatively connected with the secure element 11200, the BLE unit 11100, and the NFC target unit 11300 as shown in the figure. Definitely and apparently, the biometric unit could be also incorporated by and for use with all of the above embodiments in a similar manner.

In some embodiments such as the embodiment as shown in the FIG. 9, the active operation unit is a BLE unit 11100 comprising a plurality of contacts for receiving power from removably coupled power transmitter and the power transfer unit is a USB power transfer unit 11600 comprising a USB power transmitter, preferably equipped with connection members, such as connection pins or hooks, and/or fixing or locking members for making contact and/or interlocking with the contacts of the BLE unit, whereby providing the power to the BLE unit for conducting wireless data communication with a provisioning unit, namely a smartphone 11400 as shown in the FIG. 9. The passive operation unit is a NFC passive target unit 11300 operatively coupled with and wirelessly powered by a payment transaction unit, namely a wireless data transaction terminal 11500, and configured to draw its operating power from the payment transaction unit acting as a NFC initiator unit for conducting data verification and/or contactless data transaction operation with the data transaction terminal via the secure element 11200.

Further, in the hybrid device as shown in FIG. 9, the secure element 11200, the BLE unit 11100, and the NFC passive target unit 11300 are operatively coupled with each other and mounted on a bendable board 11700 adapted for easy mounting on a non-planar or a curved surface and/or being readily insertable or embedded into a curved accommodating space. In some embodiments, the biometric unit 11800 is also operatively coupled with the secure element 11200, the BLE unit 11100, and the NFC passive target unit 11300 and mounted together with them on the bendable board 11700. Alternatively, the BLE unit is adapted to be removably connected with the secure element without mounting fixedly on the bendable board; and/or the BLE unit might be integrated with the USB power transfer unit 11600, similar to the foregoing embodiments.

In some embodiments, the hybrid device is mounted at and preferably embedded into a band or buckle, preferably made of a non-metal material such as leather or plastic or the like to reduce the interference thereof, of a traditional watch to replace the original band or buckle of the watch of a user for conducting contactless data transactions and other applicable operations making use of biometric data, health data, hardware specific data, and/or Government related security data and the like, such that the user could wear and use the watch in a traditional way while benefit from the contactless secure data communication/processing capabilities provided by the new and replaceable part of the watch.

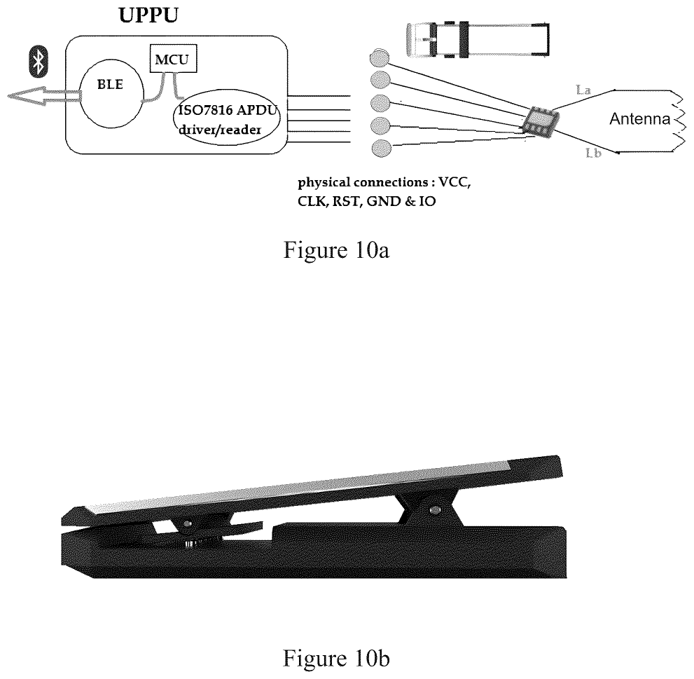

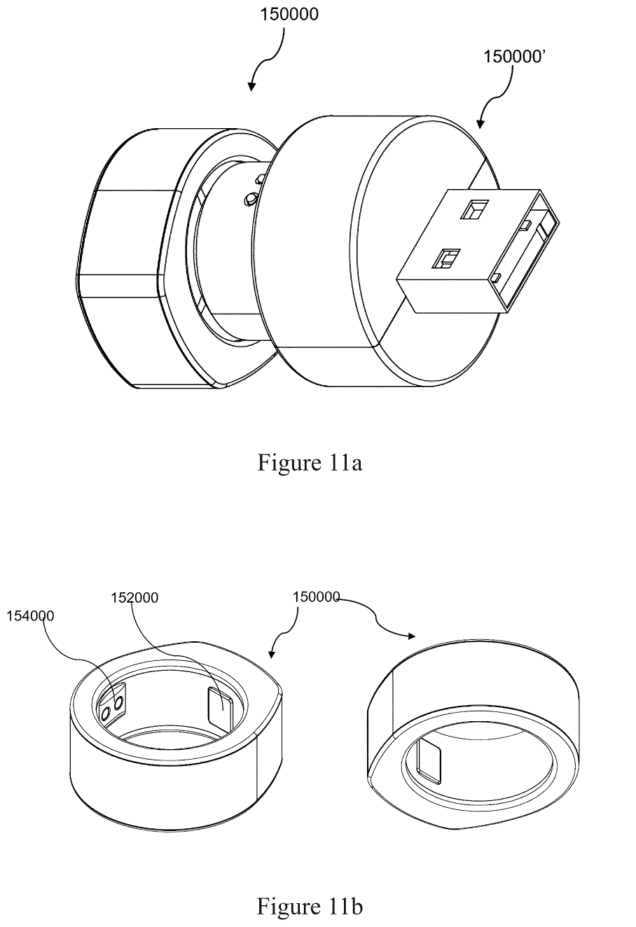

Referring to FIGS. 10a-10b, schematic views of an universal passive provisioning unit or the active operation unit adopted by the battery-less active and passive hybrid device according to one preferred embodiment of the present disclosure are illustrated, wherein the active operation unit is integrated with the power transfer unit and/or an interface for power transfer to form an universal passive provisioning unit being substantially in form of a clip/clamp or a forepart of clothes-pin and adapted for provisioning various matched and compatible smart wearables of various forms and dimensions (such us a wristband or a watch band of various dimensions as described above). The power transfer unit/the interface for power transfer of the embodiment is a USB power transmitter or a USB interface/cable with ends connected respectively with a USB power socket (e.g. the power socket on a smartphone, or a mobile device. a computer, or the like) and a proximal end (such as the rightmost end in the figure) of the universal passive provisioning unit. The universal passive provisioning unit comprises a BLE controller/unit and a ISO7816 type APDU driver/reader unit operatively connected with a MCU, which comes with a plurality of connection pins, such as 5 connection pins (namely the VCC, CLK, RST, GND, and IO pins) as shown in the FIG. 10a, for enabling a detachable connection and communication with the secure element and NFC unit mounted on a passive bendable board embedded or mounted into a smart wearble device, such as a wristband or watch band with 5 contacts as shown in the FIG. 10a. The connection pins of the universal passive provisioning unit are adapted for engaging or coupling with the 5 contacts for provisioning one or more personalization information data, biometric data, health data, government related security data, hardware specific data, and/or a token of the device to the secure element. After provisioning process, the universal passive provisioning unit will be disengaged with the bendable board, such that the passive bendable board is now adapted and suitable for conducting data verification and/or contactless data transaction operation with dedicated terminal or conventional POS terminals via the secure element.

Referring to FIG. 10b, the universal passive provisioning unit substantially in the form of a clip/clamp or a forepart of clothes-pin and adapted for provisioning various matched and compatible smart wearables of various forms and dimensions (such us a wristband or a watch band of various dimensions as described above and shown in the figure) is depicted, which is similar to the power transfer unit shown in FIGS. 6-7 in terms of their appearance and also comprises a lower part with a flattened portion located at its proximal end and a depressed portion at its distal end on which a plurality of connection members are arranged for enabling power and signal transmission; and an upper part hingeably mounted over the lower part and configured to be switchable between an opened position/state in which its proximal end is configured to make contact with the proximal end of the lower part and its distal end is configured to disengage with the distal end of the lower part, such that the connection members and depressed portion are accessible to receive at least partly a respective external engageable passive member (such as a wristband or a watch band as described above and shown in FIG. 10b) comprising the secure element and passive operation unit adapted for conducting power and data transmission or operation therebetween, such as operation for provisioning of personalization information data, biometric data, health data, government related security data, hardware specific identification data, and/or a token of the device; and a closed position/state (the position as shown in FIG. 10b) in which its distal end is configured to make contact with the distal end of the lower part and its proximal end is configured to disengage with the proximal end of the lower part, such that the connection members and depressed portion are covered by the upper part and the engaged passive member is sandwiched between and locked by the upper and lower parts for facilitating the power and data transmission or operation therebetween. As can be seen from the figure, an optional protrusion extended in a width direction is arranged at the bottom side of the upper part and positioned at a location corresponding to the same of the connection members arranged at the lower art, so as to act as an optional fixing or locking member for facilitating or securing the engagement between the the universal passive provisioning unit and the passive member, as shown in the FIG. 10b. After completion of the the power and data transmission or operation, such as operation for provisioning, the upper part might be hinged or switched to the opened position/state and the provisioned passive member could be removed from the depressed portion and is made ready and configured for conducting data verification and/or contactless data transaction operation with dedicated terminals or conventional terminals, such as a NFC POS terminal, via the secure element.

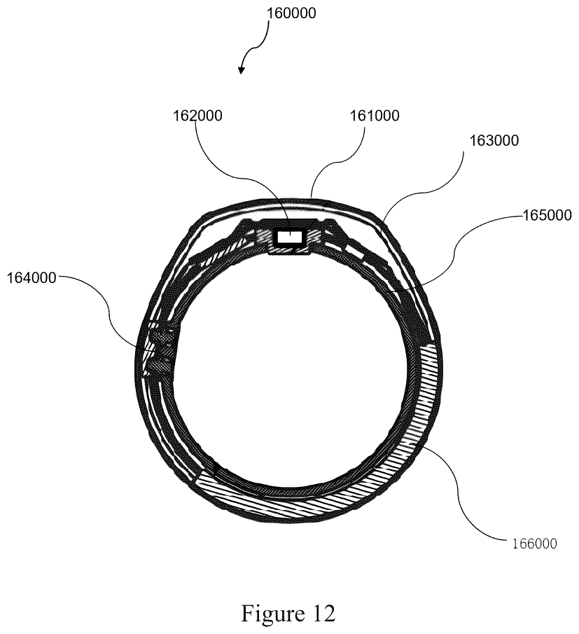

Referring to FIGS. 11a and 11b, schematic views of a token smart ring and its coupleable accessary incorporating the universal passive provisioning unit 150000′ adopted by the battery-less active and passive hybrid device according to one preferred embodiment of the present disclosures are illustrated, wherein a main portion of the universal passive provisioning unit 150000′ shown in FIG. 11a is substantially in the form of a cylinder (the first cylinder), while one end of its opposite ends is removably engaged or integrated with an interface unit, such as a conventional USB interface for power and/or data transfer, whereby provisioning or charging various matched and compatible smart wearables of various forms and dimensions, such us the token smart ring depicted in FIGS. 11a-12, upon coupling therewith. As depicted in FIG. 11b, the token smart right comprises a health monitoring sensor or unit 152000 for holding one or more sensors for sensing various biometric data from a wearer of the token smart ring and a power/data transfer or contact unit 154000 for enabling a power and/or data transaction between the token smart ring and an external removable unit connected therewith. As depicted in FIG. 11a, the power transfer unit/the interface for power transfer of the embodiment is a USB power transmitter or a USB interface/cable with ends adapted for connecting respectively with a USB power socket (e.g. the power socket on a smartphone, or a mobile device. a computer, or the like) and a proximal end, such as the rightmost end of the main portion of the universal passive provisioning unit 150000′, as described in details hereinbefore. The other end of the opposite ends is removably engaged or integrated with an engagement or charging unit substantially also in the form of a cylinder (the second cylinder) with a relatively small diameter for insertion into or coupling with the token smart ring whereby provisioning or charging it once engaged therewith. In some embodiments, wherein the first cylinder or one of its opposite ends might acts as an alignment member and/or a stopper for the token smart ring during its coupling with the second cylinder. In the embodiment, contacts are formed on the cylindrical surface of the second cylinder for matching and cooperating with the associated contacts on the inner circular surface of the token smart ring once coupling therewith, whereby enabling a detachable connection and communication with the secure element and other functional units embedded or mounted into the token smart ring. The contacts or connection pins of the universal passive provisioning unit are adapted for engaging or coupling with the associated power/data transfer or contact unit 154000 of the token smart ring for provisioning one or more personalization information data, biometric data, health data, government related security data, hardware specific data, and/or a token of the device to the secure element and/or charging an optional chargeable energy source embedded into or in electrical connection with the token smart ring. After a provisioning process and/or a charging process, the universal passive provisioning unit will be disengaged with the token smart ring, such that the token smart ring is now adapted and suitable for conducting data verification and/or contactless data transaction operation with dedicated terminal or conventional POS terminals via the secure element. Alternatively or concurrently, the health monitoring sensor or unit 152000 in the token smart ring might be activated for sensing/gathering/collecting, storing, and/or analysing a biometric data originated from the user wearing the token smart ring for conducting one or more preset or scheduled health monitoring procedures or processes.

According to the present disclosure, the token smart ring might be made of different materials comprising ceramic, plastic, and stainless steel and have a water resistant level of IPX7. In some embodiments, it comprises a battery or power source, which might be a Lithium battery with 20 mAh nominal capacity or above and could be fully charged in 90 minutes or less. It might have 3 to 5 days of battery life, which might be last for up to 6 years depending on the usage by the end user of the ring. In several embodiments, it has a width of 10 mm +/−0.5 mm or less, a thickness of 4.5 mm +/−0.5 mm or less, a weight of 6 to 8 grams or below, basically depending on size of the ring. For example, the respective weights of the ring with conventional ring size #12, #14, #18, #20, and #24 are approximately 6.6˜6.9 grams, 6.7˜7.0 grams, 6.9˜7.2 grams, 7.1˜7.4 grams, and 7.3˜7.6 grams; wherein the ring size might be adapted to the size standard of a targeted country or any other known size standard in various regions and countries. In other embodiments, the token smart ring comprises a communication unit supporting various protocols including Bluetooth 4.0 BLE, and/or-NFC, or the like.

According to the present disclosure, the token smart ring might comprise one or more sensors/monitors/meters including heart rate sensor, temperature sensor, accelerometer. It might comprise a heart rate monitor for measuring heart rate in real-time or recording the heart rate, a calories burned meter for computing total calories burned in hour or walking ranges calculation; and/or a step counting monitor for total step counting (per day/per week/per month) and total walk mileage calculation; and/or a sleep duration monitor for gathering information obtainable in 4 categories including A. Non-REM, B. Deep, C. Light, and D. Awake. In further embodiments, the token smart ring might comprise a thermometer for body temperature measurement. In some preferred embodiments, it comprises a wireless payment unit embedded a secure element or chip preferably with an antenna located inside the ring for wireless payment; and/or a data storage unit such that the data related to heart rate, calories, steps counting, sleep duration and body temperature information could be stored and display via an associated mobile APP, wherein all data record can be stored up to 14 days in the ring. In some other embodiments, the token smart ring comes with APP SDK and provide full feature's SDK information for APP development to enlarge the scope or coverage of the product.

In other embodiments, the token smart ring might accompany with a charging dongle having various indicators for providing simple charging features and indicating the current charging stage/status and operation modes of the dongle while connecting with power source and in operation, for example, a red light emitted from the dongle might means the dongle is connected with power and in operation, while a flickering green light might means the ring is currently under charging, and a green light might means the ring is fully charged. In several embodiments, the dongle engraves with size number which matches with the ring size to facilitate an easy identification when several rings are placed adjacent to one another.

Referring to FIG. 12, a schematic sectional view of the token smart ring adapted for use with the universal passive provisioning unit adopted by the battery-less active and passive hybrid device according to another preferred embodiment of the present disclosure is depicted, which is similar to the token smart ring shown in FIGS. 11a and 11b in terms of their appearance and configuration. In this embodiment of the present disclosure, which relates to a token smart ring 160000, preferably adapted for wireless payment and/or health monitoring, comprising a main portion adapted for engaging with an external associated object, such as a finger of the ring wearer or an optional accessary including a dedicated charging device accompanied therewith, and including an inner space preferably formed by a structure of a plurality of layers 161000, 163000, 165000 for receiving or mounting at least one functional unit (such as 162000, 164000, 166000); wherein the at least one functional unit including one or more of the following features:
a wireless payment or data transaction unit (162000) comprising a secure element adapted for enabling or conducting a secure wireless payment or data transaction with an external matched or associated apparatus; and/or
a health monitoring unit (162000) comprising at least one sensor and/or status monitor/controller for sensing, storing, and/or analysing a biometric data adapted and configured for health monitoring;
preferably the at least one sensor and/or status monitor/controller comprising a heart rate sensor for sensing a human heart rate; a temperature sensor for sensing ambient or surrounding temperature and/or a human body temperature; and/or an accelerometer sensor for measuring seismic activity, inclination, vibration, dynamic distance and speed of the token smart ring with or without the influence of gravity; and/or
a heart rate monitor for measuring and storing/recording heart rate in a real-time manner; and/or
a calories burned calculator for computing or estimating how many calories being burned during a predetermined routine daily human activity or a workout or exercise based on a predetermined activity duration including a day, a week, and/or a month; and/or
a pedometer or step counting monitor for measuring or computing a number of human steps and/or a human travel distance or a walk mileage; and/or for tracking or recording multiple metrics including a heart rate, a sleep status, a stress status, a blood oxygen level, a calories burned value, and/or an activity duration; and/or
a sleep status monitor for computing or recording a sleep status data including a wake up time or a bed time based on a preset duration of sleep required, whereby facilitating a determination of an optimal sleep schedule based on a human unique situation including an age, a wake up time and/or a bed time; and preferably the sleep status data including a data obtained respectively in a Non-REM (rapid eye movement) sleep stages including awake, light, and deep sleep stages, and a REM (rapid eye movement) sleep stage of a sleep; and/or a thermometer adapted for a body temperature measurement; and/or
a data storage unit adapted for storing and presenting a predetermined set of data including a heart rate data, a calorie data, a step counting data, a sleep duration data, and/or a body temperature data; and preferably the predetermined set of data is configured to be stored up to and/or valid through at least 14 days.

In some embodiments, the token smart ring might further comprise a foregoing biometric unit for secondary or biometric authentication of user identity comprising one or more biometric readers/sensors capable of reading, writing and processing biometric data related to vein pattern, fingerprints, hand geometry, DNA, voice pattern, iris pattern, signature dynamics and/or face detection. The biometric unit might be operatively connected with the secure element, the wireless payment or data transaction unit, and/or the health monitoring unit, as the biometric unit might be incorporated by and for use with the embodiments of the token smart ring in a similar manner as described hereinbefore. In other embodiments, the token smart ring is configured to be switchable between a health monitoring state and a secure data transaction state; wherein in the health monitoring state the health monitoring unit is activated and configured for sensing, storing, and/or analysing the biometric data originated or came from an engageable portion of the external associated object for facilitating a human health monitoring and/or promoting operation; while in the secure data transaction state the health monitoring unit might be deactivated and the wireless payment or data transaction unit and/or the secure element is activated to make the token smart ring to act as an active device adapted for provisioning one or more personalization information data, biometric data, health data, government related security data, hardware specific data, and/or a token of the smart ring to the secure element or a passive device adapted for conducting wireless secure payment, data verification and/or contactless data transaction operation, with a preset or compatible external device as described or set forth hereinbefore, via the secure element.

In some embodiments, wherein the main portion substantial comprised or made of a plurality of layers including three layers preferably of different materials, and preferably including an innermost layer substantially made of ceramic adapted for engaging the ring with the external associated object, an intermediate layer substantially made of stainless steel configured for mounting the at least one functional unit at the ring, and an outer layer substantially made of plastic adapted for coupling with the innermost layer to form the inner space for receiving the intermediate layer and the at least one functional unit. The structure of multi-layers might provide a desired wearing comfortability, stiffness, and anti-interference capability of the ring.

In some other embodiments, wherein the wireless payment or data transaction unit is a battery-less active and passive hybrid device for secure wireless data transaction comprising a secure element adapted for storing at least one secure applet and at least one data segment for secure contactless data transaction and operatively connected with an active operation unit and a passive operation unit; and preferably the active operation unit is adapted to be connected removably with the secure element; and wherein the hybrid device is configured to be switchable between an active state in which the active operation unit is activated on demand to enable the hybrid device to function as an active device and adapted for provisioning one or more personalization information data, biometric data, health data, government related security data, hardware specific data, and/or a token of the device to the secure element; and a passive state in which the active operation unit is deactivated and the passive operation unit is activated/configured to enable the hybrid device to function as a passive device and adapted for conducting data verification and/or contactless data transaction operation via the secure element.

In several embodiments of the token smart ring according to the present disclosure, wherein it is configured to have a water resistant level of IPX7; and/or it further comprises a power unit 166000 adapted for powering the token smart ring to conduct a desired operation for at least 3 days and lasting for up to 6 years depending on usage of the smart ring or frequency of the desired operation, and preferably comprises a data transfer interface and/or a power charging interface for engaging with an external associated transfer device for conducting a data transfer and/or a power transfer therebetween.

In other embodiments, wherein the data transfer interface and/or the power charging interface 164000 comprises a female type connector with a plurality of female contacts hidden from view for receiving or removably coupling with a male type connector of the external associated transfer device with an outer dimension or an outer profile in line with an inner profile of the main portion for forming a removable tight fit with the token smart ring for conducting data or power transfer therewith.

In further embodiments, wherein the female type connector comprises a rounded edge female contact substantially in form a circular hole; and the male type connector comprises a rounded edge male contact substantially in form a circular pin being movable along or in a radial direction and insertable into or removable from the circular hole when engaging or disengaging with the female type connector; and/or the external associated transfer device has an round outer dimension being insertable into or removable from an inner cavity of the main portion for conducting or switching off a data or power transfer therewith.

In other embodiments, wherein the female type connector and/or the external associated transfer device comprise a visual and/or audio indicator for indicating a working state or status and/or a working result of a data or power transfer operation carried out therebetween.

In additional embodiments, further comprising a WIFI, BT including BLE, and/or NFC enabled communication unit for conducting wireless secure data transaction and/or wireless secure payment with the external matched or associated apparatus; and preferably the communication unit is configured for conducting wireless secure payment with an external matched or associated POS terminal.

In some other embodiments, wherein the associated transfer device comprises a first cylindrical part substantially in the form of a cylinder, while one end of its opposite ends is removably engaged or integrated with an interface unit for power and/or data transfer whereby enabling a provisioning operation and/or a charging operation upon coupling with the token smart ring; wherein another end of the opposite ends is removably engaged or integrated with a second cylindrical part substantially also in the form of a cylinder with a diameter small than that of the first cylindrical part and adapted for insertion into or coupling with the token smart ring whereby provisioning or charging it once engaged therewith; wherein the first cylindrical part acts as an alignment member and/or a stopper for the token smart ring during its coupling with the second cylindrical part.

In some embodiments, wherein it has a width of 10 mm +/−0.5 mm or less, a thickness of 4.5 mm +/−0.5 mm or less, and/or a weight of 6-8 grams or less; wherein each of above parameters depends on the applicable size of the token smart ring.

The functional unit (162000) including the wireless payment or data transaction unit and/or the health monitoring unit might be preferably located at a top end of the token smart ring (as depicted in FIG. 12), which protrudes outwardly while in position for facilitating the secure wireless payment or data transaction conducted with an external matched or associated apparatus and/or the collecting of the biometric data from the ring wearer for health monitoring.

The present disclosure is described according to specific embodiments, but those skilled in the art will appreciate that various changes and equivalents might be made without departing from the scope of the present disclosure. In addition, many modifications might be made to the present disclosure without departing from the scope of the invention in order to adapt to specific circumstances or components of the present disclosure. Accordingly, the present disclosure is not limited to the specific embodiments disclosed herein, and shall include all embodiments falling within the scope of the claims.

1. A token smart ring, preferably adapted for wireless payment and/or health monitoring, comprising a main portion adapted for engaging with an external associated object and including an inner space preferably formed by a structure of a plurality of layers for receiving or mounting at least one functional unit; wherein the at least one functional unit including:
a wireless payment or data transaction unit comprising a secure element adapted for enabling or conducting a secure wireless payment or data transaction with an external matched or associated apparatus; and/or
a health monitoring unit comprising at least one sensor and/or status monitor/controller for sensing, storing, and/or analysing a biometric data adapted and configured for health monitoring;
preferably the at least one sensor and/or status monitor/controller comprising a heart rate sensor for sensing a human heart rate; a temperature sensor for sensing ambient or surrounding temperature and/or a human body temperature; and/or an accelerometer sensor for measuring seismic activity, inclination, vibration, dynamic distance and speed of the token smart ring with or without the influence of gravity; and/or
a heart rate monitor for measuring and storing/recording heart rate in a real-time manner; and/or
a calories burned calculator for computing or estimating how many calories being burned during a predetermined routine daily human activity or a workout or exercise based on a predetermined activity duration including a day, a week, and/or a month; and/or
a pedometer or step counting monitor for measuring or computing a number of human steps and/or a human travel distance or a walk mileage; and/or for tracking or recording multiple metrics including a heart rate, a sleep status, a stress status, a blood oxygen level, a calories burned value, and/or an activity duration; and/or
a sleep status monitor for computing or recording a sleep status data including a wake up time or a bed time based on a preset duration of sleep required, whereby facilitating a determination of an optimal sleep schedule based on a human unique situation including an age, a wake up time and/or a bed time; and preferably the sleep status data including a data obtained respectively in a Non-REM (rapid eye movement) sleep stages including awake, light, and deep sleep stages, and a REM (rapid eye movement) sleep stage of a sleep; and/or
a thermometer adapted for a body temperature measurement; and/or
a data storage unit adapted for storing and presenting a predetermined set of data including a heart rate data, a calorie data, a step counting data, a sleep duration data, and/or a body temperature data; and preferably the predetermined set of data is configured to be stored up to and/or valid through at least 14 days; and/or
a biometric unit operatively connected with the secure element, the wireless payment or data transaction unit, and/or the health monitoring unit and adapted for biometric authentication of user identity comprising one or more biometric readers/sensors capable of reading, writing and processing biometric data related to vein pattern, fingerprints, hand geometry, DNA, voice pattern, iris pattern, signature dynamics and/or face detection;
and preferably the token smart ring is configured to be switchable between a health monitoring state and a secure data transaction state; wherein in the health monitoring state the health monitoring unit is activated and configured for sensing, storing, and/or analysing the biometric data originated or came from an engageable portion of the external associated object for facilitating a human health monitoring and/or promoting operation; while in the secure data transaction state the health monitoring unit is deactivated and the wireless payment or data transaction unit and/or the secure element is activated to make the token smart ring to act as an active device adapted for provisioning one or more personalization information data, biometric data, health data, government related security data, hardware specific data, and/or a token of the smart ring to the secure element or a passive device adapted for conducting wireless secure payment, data verification and/or contactless data transaction operation via the secure element.

2. The token smart ring of claim 1, wherein the main portion substantial comprised or made of a plurality of layers including three layers preferably of different materials, and preferably including an innermost layer substantially made of ceramic adapted for engaging the ring with the external associated object, an intermediate layer substantially made of stainless steel configured for mounting the at least one functional unit at the ring, and an outer layer substantially made of plastic adapted for coupling with the innermost layer to form the inner space for receiving the intermediate layer and the at least one functional unit.

3. The token smart ring of claim 1, wherein the wireless payment or data transaction unit is a battery-less active and passive hybrid device for secure wireless data transaction comprising a secure element adapted for storing at least one secure applet and at least one data segment for secure contactless data transaction and operatively connected with an active operation unit and a passive operation unit; and preferably the active operation unit is adapted to be connected removably with the secure element; and wherein the hybrid device is configured to be switchable between an active state in which the active operation unit is activated on demand to enable the hybrid device to function as an active device and adapted for provisioning one or more personalization information data, biometric data, health data, government related security data, hardware specific data, and/or a token of the device to the secure element; and a passive state in which the active operation unit is deactivated and the passive operation unit is activated/configured to enable the hybrid device to function as a passive device and adapted for conducting data verification and/or contactless data transaction operation via the secure element.

4. The token smart ring of claim 1, wherein it is configured to have a water resistant level of IPX7; and/or it further comprises a power unit adapted for powering the token smart ring to conduct a desired operation for at least 3 days and lasting for up to 6 years depending on usage of the smart ring or frequency of the desired operation, and preferably comprises a data transfer interface and/or a power charging interface for engaging with an external associated transfer device for conducting a data transfer and/or a power transfer therebetween.

5. The token smart ring of claim 4, wherein the data transfer interface and/or the power charging interface comprises a female type connector with a plurality of female contacts hidden from view for receiving or removably coupling with a male type connector of the external associated transfer device with an outer dimension or an outer profile in line with an inner profile of the main portion for forming a removable tight fit with the token smart ring for conducting data or power transfer therewith.

6. The token smart ring of claim 5, wherein the female type connector comprises a rounded edge female contact substantially in form a circular hole; and the male type connector comprises a rounded edge male contact substantially in form a circular pin being movable along or in a radial direction and insertable into or removable from the circular hole when engaging or disengaging with the female type connector; and/or the external associated transfer device has an round outer dimension being insertable into or removable from an inner cavity of the main portion for conducting or switching off a data or power transfer therewith.

7. The token smart ring of claim 6, wherein the female type connector and/or the external associated transfer device comprise a visual and/or audio indicator for indicating a working state or status and/or a working result of a data or power transfer operation carried out therebetween.

8. The token smart ring of claim 1, further comprising a WIFI, BT including BLE, and/or NFC enabled communication unit for conducting wireless secure data transaction and/or wireless secure payment with the external matched or associated apparatus; and preferably the communication unit is configured for conducting wireless secure payment with an external matched or associated POS terminal.

9. The token smart ring of claim 4, wherein the associated transfer device comprises a first cylindrical part substantially in the form of a cylinder, while one end of its opposite ends is removably engaged or integrated with an interface unit for power and/or data transfer whereby enabling a provisioning operation and/or a charging operation upon coupling with the token smart ring; wherein another end of the opposite ends is removably engaged or integrated with a second cylindrical part substantially also in the form of a cylinder with a diameter small than that of the first cylindrical part and adapted for insertion into or coupling with the token smart ring whereby provisioning or charging it once engaged therewith; wherein the first cylindrical part acts as an alignment member and/or a stopper for the token smart ring during its coupling with the second cylindrical part.

10. The token smart ring of claim 1, wherein it has a width of 10 mm +/−0.5 mm or less, a thickness of 4.5 mm +/−0.5 mm or less, and/or a weight of 6-8 grams or less.
